SEQUENCE LISTING (1 sequence)

SEQ ID NO: 1 - DNA, 535 bp, Bacillus amyloliquefaciens
tggagagtttgatcctggctcaggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtttgaaccgcatggttcagacataaaaggtggcttcggctaccacttacagatggacccgcggcgcattagctagttggtgaggtaacggctcaccaaggcracgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggta

(19) United States
(12) Patent Application Publication
(10) Pub. No.: US 2015/0258150 A1
(43) Pub. Date: Sep. 17, 2015

(54) BACILLUS AMYLOLIQUEFACIENS STRAIN

(71) Applicant: OSPREY BIOTECHNICS, INC., SARASOTA, FL (US)

(72) Inventors: Christopher J. Reuter, Parrish, FL (US); Steven J. MacKenzie, Sarasota, FL (US); Lauren G. Danielson, Bradenton, FL (US); Vincent Scuilla, Sarasota, FL (US)

(73) Assignee: Osprey Biotechnics, Inc., Sarasota, FL (US)

(21) Appl. No.: 14/211,078

(22) Filed: Mar. 14, 2014

(51) Int. Cl. A61K 35/74; A01N 63/00

(52) U.S. Cl. CPC A61K 35/741; A01N 63/00

(57) ABSTRACT

A novel strain of Bacillus amyloliquefaciens is effective in inhibiting the growth and activity of gram negative bacteria that can present health and safety problems for food processing, agriculture and other areas.

### CROSS-REFERENCE TO RELATED APPLICATIONS

Not Applicable.

### FIELD OF THE DISCLOSURE

This disclosure concerns a novel Bacillus amyloliquefaciens strain useful for inhibiting the growth of gram negative microorganisms.

### BACKGROUND OF THE DISCLOSURE

Bacillus amyloliquefaciens is a gram-positive non-pathogenic soil bacterium similar to other Bacillus species, and closely related to Bacillus subtilus. Bacillus amyloliquefaciens is known for its ability to degrade proteins extracellularly. Enzyme, subtilisin, secreted by Bacillus amyloliquefaciens has been used in laundry detergents and contact lens cleansers.

E. coli and Salmonella present problems as certain pathogenic strains of these organisms produce extraordinary toxins. Cattle and swine can be a reservoir for these pathogenic strains and the animals can shed these organisms into the environment. In fact, there are often high incidences of contamination of feedlots and pens. Infections in humans usually are the result of undercooked meat or raw vegetables. Since E. coli and Salmonella are both persistent organisms that survive well in water and moist environments, the human food supply is often infected from animal feces either through runoff into the water supply where said water is used to water vegetable crops or through processing of meat. Therefore, it is important to health of both people and animals to provide new strains that have the capability to eliminate the pathogenic E. coli and Salmonella strains in the environment and in farm animals to reduce the incidence of disease, to increase productivity and to decrease the incidence of death due to the pathogenic organism.

Plants are affected by a variety of gram negative pathogens. Some of the pathogens are soil borne and others are foliar. Some of the most troublesome plant pathogen diseases caused by gram negative bacteria include Ralstonia solanacearum, which causes bacterial wilt, Pseudomonas syringae, which causes diseases like bacterial blight and bacterial brown spot, and a variety of Xanthanomas species, which causes a variety of plant diseases including bacterial blights and spots and citrus canker. While other strains have shown antibacterial activity in the literature, new and effective strains showing antibacterial activity against specific diseases are desired for producing new biopesticidal products.

Pathogenic microorganisms cause major losses in aquaculture farming. Several Vibrio species are pathogenic to fish and shellfish. These pathogens cause infection, resulting in a loss of growth and productivity, as well as mortality, particularly to the larvae. This results in a serious financial hurdle for the aquaculture industry. Thus, it is important to provide new and effective strains that have antibacterial activity against specific disease-causing microbes affecting the aquaculture industry.

### SUMMARY OF THE DISCLOSURE

A novel strain of Bacillus amyloliquefaciens has been identified and isolated, namely Bacillus amyloliquefaciens strain OBT712. This strain is characterized by its ability to inhibit the growth and/or activity of gram negative microorganisms, such as E. coli, Salmonella, Ralstonia solanacearum, Pseudomonas, Xanthanomas, Vibrio and others.

The novel strain of Bacillus amyloliquefaciens can be used in numerous applications involving contacting a gram negative pathogen with an effective amount of the Bacillus amyloliquefaciens strain OBT712 to provide an inhibiting effect.

### BRIEF DESCRIPTION OF THE DRAWING

FIG. 1 is the relevant sequence (SEQ. ID NO. 1) for Bacillus amyloliquefaciens strain OBT712.

FIG. 2 is a graph illustrating Toxin CDT-A and CDT-B concentration of two Clostridium cultures that are identical except for the addition of OBT712 to one of the samples.

FIG. 3 is a graph illustrating bacteria populations or concentrations for two Clostridium cultures that are identical except for the addition of OBT712 to one of the two samples.

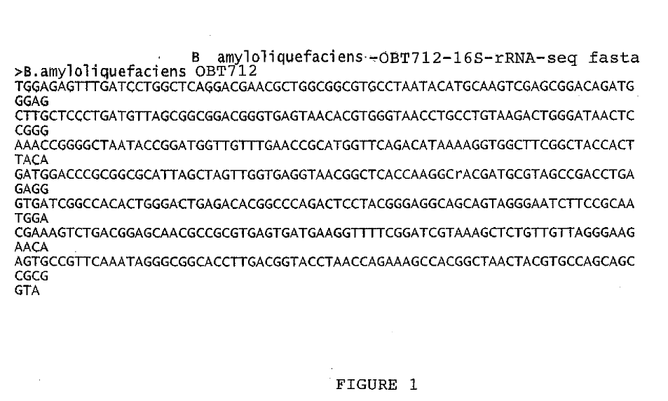

### DETAILED DESCRIPTION

The novel OBT712 strain produces substances, such as enzymes, metabolites, and/or antibiotics, which are effective in inhibiting the growth of gram negative bacteria, such as E. coli, Salmonella, Ralstonia solanacearum, Pseudomonas, Xanthanomas, Vibrio and other gram negative pathogenic microorganisms. Gram negative pathogens present a problem in numerous areas from food processing, to health, to growth productivity and morbidity of livestock and poultry, to causing disease and thereby affecting the productivity of plants.

The term “inhibit” means to reduce or arrest growth and/or reproduction of gram negative bacteria, and/or to reduce or arrest production by and/or secretion of toxins from gram negative bacteria. The term “inhibit” also encompasses killing gram negative bacteria and/or reducing or arresting the pathogenic or toxic effects of gram negative bacteria on humans or animals.

The term “effective amount” means an amount that will achieve a desired inhibiting effect to reduce incidents of disease caused by gram negative bacterial pathogens. Effective amounts for particular applications can be determined by routine experimentation.

The Bacillus amyloliquefaciens strain OBT712 and its inhibitory effect on gram negative pathogens have a number of applications. These include: improving food safety and thereby reducing incidents of disease in humans; treating wastewater and food processing plants to reduce incidents of disease in humans and animals; treating feedlot, holding pens, animal feed as a direct fed microbial or in drinking water and manure piles of livestock to reduce the incidence of disease to the animal and to humans; treating ponds and water retention facilities where farm runoff may infect the waters and where such waters may be put to other uses, such as watering vegetable crops that may be eaten raw by humans; as a direct fed microbial to reduce incidents of disease in animals; as a biopesticide against gram negative plant pathogens; and in aquaculture to reduce disease and mortality from gram negative microorganisms.

Wastewater contaminated with E. coli or Salmonella can be treated with the novel OBT712 by spraying, direct inoculation of a liquid or a powder or a block containing the bacteria to reduce the E. coli and/or Salmonella and other gram negative.

Waste lagoons, manure piles, feedlots or pens containing waste from pig, cattle, sheep, chicken and equine can be treated with the novel OBT712 by spraying or dispersing in a powder form the bacteria to reduce the E. coli, Salmonella and other gram negative species.

Animal feed or drinking water can be treated by using the novel OBT712 bacteria as a direct fed microbial food supplement to reduce intestinal E. coli and Salmonella and other pathogenic gram negative species.

Meat or other food processing facility waste can be treated with the novel OBT712 by spraying, direct inoculation of a liquid or a powder or a block containing the bacteria to reduce the E. coli and/or Salmonella or other pathogenic gram negative bacterial contamination.

Farm ponds or ponds or other water sources that may be subject to sewage or manure runoff can be treated with the novel OBT712 by spraying, direct inoculation of a liquid or a powder or a block containing the bacteria to reduce the E. coli, Salmonella or other gram negative bacteria.

The OBT712 strain may be used to reduce biofilms that are formed by pathogenic gram negative organisms by applying the OBT712 to the biofilm, through a spray, a powder or liquid or by suspending the same in a block that is submerged to reach the biofilm.

The novel OBT712 bacterium can be used for the treatment of gram negative pathogens on plants, as a seed treatment, a root dip, a soil treatment, or a foliar spray either in a spray or by inoculating water that may be used in a sprinkler or drip irrigation system or applied via spray from a truck, as a soil drench or by direct application to the seed or roots, either in a liquid or powder form, in order to reduce Ralstonia solanacearum, Pseudomonas syringae, Xanthanomas species and other like gram negatives that act as a plant pathogens, thereby increasing the productivity of the plant.

The novel OBT712 bacteria can be used for the reduction of Vibrio pathogens in the aquaculture industry. The OBT712 bacteria can be added directly to the tank or pond water or added to the feed where the shrimp or fish are being farmed. This treatment can be done with a liquid or block in the tank or pond, or can be provided to the rotifers, which then become the food for the fish or shrimp species. The treatment can be done either in the form of a spore or in a live vegetative state. This treatment can help to reduce the incidence of Vibrio in the larval brooding tanks, or to reduce or suppress Vibrio on multiple salt-water or shell fish that are grown in farming. The OBT712 also produces the enzyme proteinase, which may assist in providing waste reduction in the tanks or ponds.

The disclosed Bacillus amyloliquefaciens strain OBT712 can be biologically purified and/or provided in a dried, sporulated form (e.g., as a powder or particulate), or in a liquid carrier composition. Such liquid or dry compositions can comprise various additives such as nutrients, dispersants, stabilizers, fragrances and dyes.

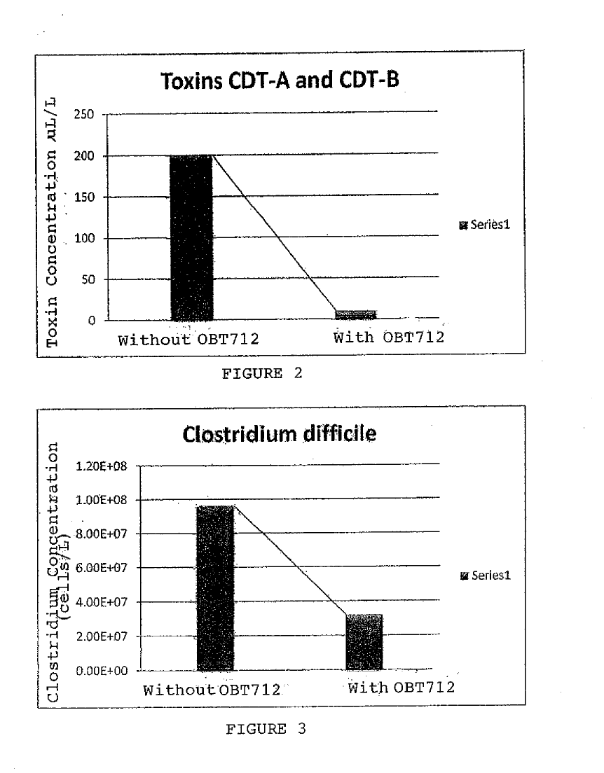

The effect of adding OBT712 to a Clostridium culture producing CDT-A and CDT-B toxins is shown in FIG. 2. The addition of the Bacillus amyloliquefaciens strain OBT712 reduces CDT-A and CDT-B toxin production to a substantially lower level.

The effect of adding OBT712 to a Clostridium culture population is shown in FIG. 3. The addition of Bacillus amyloliquefaciens strain OBT712 profoundly reduces the Clostridium bacteria population.

The described embodiments are preferred and/or illustrated, but are not limiting. Various modifications are considered within the purview and scope of the appended claims.

1-8. (canceled)

9. A method of inhibiting gram negative microorganisms, comprising:
adding to a material contaminated with a pathogenic gram negative microorganism an amount of Bacillus amyloliquefaciens strain OBT712 as deposited with the American Type Culture Collection under accession number PTA-122189 that produces enzymes, metabolites or antibiotics that inhibit growth of gram negative pathogenic microorganisms that is effective to inhibit the gram negative microorganism.

10. The method of claim 9, in which the Bacillus amyloliquefaciens strain OBT712 is added to water contaminated with the gram negative microorganism.

11. The method of claim 9, in which the Bacillus amyloliquefaciens strain OBT712 is added to manure contaminated with the gram negative microorganism.

12. The method of claim 9, in which the Bacillus amyloliquefaciens strain OBT712 is added to animal feed or water to reduce disease in an animal.

13. The method of claim 9, in which the Bacillus amyloliquefaciens strain OBT712 is added to animal litter to reduce disease in an animal.

14. The method of claim 9, in which the Bacillus amyloliquefaciens strain OBT712 is applied to plants, seeds or soil in which plants are grown to inhibit gram negative bacteria that act as plant pathogens.

15. The method of claim 9, in which the Bacillus amyloliquefaciens strain OBT712 is applied to containments in which shrimp or fish are farmed to reduce incidents of Vibrio.

16. A composition comprising:
a mixture of Bacillus amyloliquefaciens strain OBT712 as deposited with the American Type Culture Collection under accession number PTA-122189 that produces enzymes, metabolites or antibiotics that inhibit growth of gram negative pathogenic microorganisms and at least one additive selected from dispersants, stabilizers, fragrances and dyes.

17. The composition of claim 16 in a dry form.

18. The composition of claim 16 in a liquid carrier.

19. The Bacillus amyloliquefaciens strain OBT712 of claim 1 having nucleotide Sequence ID No. 1.

20. The method of claim 9 wherein the Bacillus amyloliquefaciens strain OBT712 has nucleotide Sequence ID No. 1.

21. A method of reducing Vibrio pathogen populations in shrimp or fish, comprising:
adding to a tank or pond in which shrimp or fish are raised Bacillus amyloliquefaciens strain OBT712 as deposited with the American Type Culture Collection under accession number PTA-122189 that produces enzymes, metabolites or antibiotics that inhibit growth of gram negative pathogenic microorganisms, in an amount effective to reduce the Vibrio population in the tank or pond.
